(19) United States
(12) Patent Application Publication
(10) Pub. No.: US 2014/0187863 A1
(43) Pub. Date: Jul. 3, 2014

(54) Endoscope Controlling Device

(71) Applicant: METAL INDUSTRIES RESEARCH & DEVELOPMENT CENTRE, Kaohsiung City (TW)

(72) Inventors: Cheng-Wen Lin, Kaohsiung City (TW); Wei-Jen Shih, Kaohsiung City (TW); Yen-Nien Chen, Kaohsiung City (TW); Tzyy-Ker Sue, Kaohsiung City (TW); Pei-Yuan Lee, Kaohsiung City (TW); Hung-Wen Huang, Kaohsiung City (TW); Chien-Sheng Lo, Kaohsiung City (TW)

(73) Assignee: METAL INDUSTRIES RESEARCH & DEVELOPMENT CENTRE, Kaohsiung City (TW)

(21) Appl. No.: 13/729,093

(22) Filed: Dec. 28, 2012

(51) Int. Cl. A61B 1/00; A61B 1/04; A61B 1/005

(52) U.S. Cl. CPC A61B 1/00066; A61B 1/0058; A61B 1/04; A61B 1/00009; A61B 1/00045; A61B 1/00183; A61B 1/0052; A61B 1/00096
USPC 600/131

(57) ABSTRACT

An endoscope controlling device includes a rigid tube mounted to an end face of a base and in communication with a compartment in the base. A positioning groove is formed in an inner periphery of the base and includes a bottom wall having a slot. A linear displacement control module includes a control member and a movable board. The control member is aligned with a through-hole in a periphery of the base. The control member controls longitudinal movement of the movable board in the compartment along a longitudinal axis of the base via a driving gear. An optical element receiving drum is mounted to the movable board. A rotational movement control module is mounted around the optical element receiving drum and received in the positioning groove of the base. A portion of the rotational movement control module is extended through the slot and exposed outside of the base.

### BACKGROUND OF THE INVENTION

1. Field of the Invention

The present invention relates to an endoscope controlling device and, more particularly, to an endoscope controlling device for controlling telescopic movement and rotational movement of an endoscope.

2. Description of the Related Art

FIG. 1 shows a conventional endoscope 8 allowing rotation in two or four directions. The endoscope 8 includes a plurality of spaced hollow rotary members 81 each having a plurality of through-holes 811 (see FIG. 1a as an enlarged part A) and two connecting faces 812. The endoscope 8 further includes a plurality of connecting members 82 (see FIG. 1b as an enlarged part B) each having two ends respectively connected to two mutually facing connecting faces 812 respectively of two adjacent rotary members 81. The endoscope 8 further includes a plurality of guiding lines 83 extending through the through-holes 811 of the hollow rotary members 81. By pulling one of the guiding lines 83, the plurality of rotary members 81 can be bent in either of two directions. An example of such an endoscope 8 is disclosed in Taiwan Utility Model No. M400299.

To obtain a certain bending effect of the endoscope 8, each rotary member 81 must be small for the purposes of increasing the number of the serially connected rotary members 81. However, the difficulties in formation of the through-holes 811 in each rotary member 81 and connection of each connecting member 82 between two adjacent rotary members 81 will be aggravated by the decrease in the size of the rotary members 81 and lead to an increase in the manufacturing costs.

Furthermore, operation for bending the rotary members 81 in the desired direction requires pulling of the corresponding guiding line 83 and precise control of the magnitude of force pulling the guiding line 83, which is not easy. Further, the number of the guiding lines 83 has to be increased if the number of the directions to be controlled is increased, complicating the operation. Namely, an operator has to spend more time practicing pulling of the correct guiding line 83 and controlling the force for pulling the guiding line 83 so as to move the endoscope 8 to an ideal position in the cavity of the human body for capturing images.

FIG. 2 shows an endoscope 9 disclosed in Taiwan Patent Application No. 100143086 filed by the inventor of the present invention. The endoscope 9 includes a tube 91 having a compartment 911 extending in a longitudinal direction. A flexible strip 92 is slideably received in the compartment 911. An image capturing module 93 is mounted to the flexible strip 92 for transmitting light beams and capturing images. A controller 94 is mounted to an end of the tube 91 and connected to the flexible strip 92. The controller 94 is operable to move the flexible strip 92 relative to the tube 91.

The flexible strip 92 is made of a super elastomer (such as a shape memory alloy) having an original shape before deformation, with the super elastomer capable of restoring the original shape after large deformation. Thus, the extended length of the flexible strip 92 outside of the tube 91 can be controlled. Furthermore, the tube 91 can be rotated to arbitrarily change the visual angle of the endoscope 9. Thus, the endoscope 9 has no dead angles in obtaining images, enhancing the utility of the endoscope 9. Furthermore, the flexible strip 92 is simple in structure to significantly reduce the manufacturing costs while enhancing operational convenience.

Specifically, the controller 94 includes a hollow body 941 through which the flexible strip 92 extends. The body 941 includes a sliding groove 942 and a control member 943 slideably received in the sliding groove 942. The body 941 is fixed to the tube 91 with the sliding groove 942 in communication with the compartment 911. The control member 943 is connected to the flexible strip 92 to control telescopic movement of the flexible strip 92 relative to the tube 91. By rotating the whole controller 94, the tube 91 and the flexible strip 92 are synchronously rotated to change the image capturing angle (i.e., the visual angle).

However, in regard to the telescopic structure comprised of the flexible strip 92 relating to the tube 91, since the control member 943 and the flexible strip 92 are connected and move synchronously, the displacement of the flexible strip 92 relative to the tube 91 is the same as the displacement of the control member 943 relative to the body 941. In a case of long displacement of the control member 943, the operator has to continuously change the position of his or her palm holding the body 941. Namely, the operator can not maintain the palm in the same position and use fingers to slide the control member 943.

On the other hand, the endoscope 9 does not include devices for changing the image capturing angle. Namely, the whole controller 94 must be rotated to change the visual angle, which is difficult to precision control and minor adjustment of the image capturing angle. Thus, improvement to the operational convenience of the endoscope 9 is required.

### SUMMARY OF THE INVENTION

An objective of the present invention is to provide an endoscope controlling device to allow a user to control telescopic movement or rotational movement of an image capturing lens of an endoscope by fingers of a hand of the user without changing the position of a palm of the hand of the user during operation of the endoscope.

Another objective of the present invention is to provide an endoscope controlling device allowing minor adjustment in the telescopic movement or rotational movement of the image capturing lens, enhancing the controlling precision.

The present invention fulfills the above objectives by providing an endoscope controlling device including a base having a compartment. A rigid tube is mounted to an end face of the base and in communication with the compartment. The base further includes an inner periphery having a positioning groove. The positioning groove includes a bottom wall having a slot. The base further includes a periphery having a through-hole. A linear displacement control module includes a control member and a movable board. The control member is aligned with the through-hole of the base. The control member includes a driving gear. The control member controls longitudinal movement of the movable board in the compartment along a longitudinal axis of the base via the driving gear. An optical element receiving drum is mounted to the movable board. A rotational movement control module is mounted around the optical element receiving drum and received in the positioning groove of the base. A portion of the rotational movement control module is extended through the slot and exposed outside of the base.

Preferably, the linear displacement control module further includes a fixed board received in the compartment and connected to the base. The fixed board includes a surface facing the through-hole of the base. The movable board is slideably mounted to the fixed board.

Preferably, the surface of the fixed board includes an axle coupling seat having a peripheral wall with an opening. The axle coupling seat includes a first axle coupling portion. A driven gear is rotatably mounted to the first axle coupling portion. The fixed board further includes a second axle coupling portion. The second axle coupling portion is located adjacent to the axle coupling seat and aligned with the opening. The first axle coupling portion defines a first rotating axis, and the second axle coupling portion defines a second rotational axis parallel to the first rotating axis.

Preferably, the second axle coupling portion is aligned with the through-hole of the base, and the driving gear extends through the opening of the axle coupling seat and meshes with the driven gear.

Preferably, two sliding tracks are provided on the other surface of the fixed board opposite to the surface. Each of the sliding tracks has an opening. The openings of the sliding tracks face each other. The movable board includes a sliding plate slideably received in the sliding tracks of the fixed board.

Preferably, the sliding plate includes a toothed portion meshed with the driven gear.

Preferably, the sliding plate includes a first side having a groove. The toothed portion is formed on a lateral wall of the groove. The driven gear is received in the groove.

Preferably, the sliding plate further includes a second side opposite to the first side. An abutment portion, a retaining portion, and a tube holding portion are provided on the second side of the sliding plate along the longitudinal axis of the base. The retaining portion is located between the abutment portion and the tube holding portion. The optical element receiving drum includes a barrel, a neck, and a head. The abutment portion of the sliding plate abuts against an end of the barrel. The neck is engaged with and positioned by the retaining portion of the sliding plate. The head extends between the retaining portion and the tube holding portion.

Preferably, the barrel of the optical element receiving drum includes an outer periphery having at least one engagement portion. The rotational movement control module includes a ring and an operative ring. The ring includes an inner periphery having at least one engagement section. The at least one engagement section is engaged with the at least one engagement portion of the barrel. The operative ring is formed on an outer periphery of the ring. The ring is received in the positioning groove of the base. The operative ring is extended through the slot and exposed outside of the base.

Preferably, the control member further includes a disc and an axle. The disc is mounted to an end of the axle. The driving gear is provided on the other end of the axle. The axle is extended through the through-hole of the base and mounted around the second axle coupling portion of the fixed board. The disc is exposed outside of the base.

The endoscope controlling device converts a rotational torque into linear displacement, allowing the user to control telescopic movement or rotational movement of the image capturing lens by simply using his or her fingers without changing the position of his or her palm, providing enhanced operational convenience.

The endoscope controlling device allows minor adjustment of the telescopic movement or rotational movement of the image capturing lens through fingers that provide more precision than the wrist, providing enhanced control precision.

The present invention will become clearer in light of the following detailed description of illustrative embodiments of this invention described in connection with the drawings.

### BRIEF DESCRIPTION OF THE DRAWINGS

The illustrative embodiments may best be described by reference to the accompanying drawings where:

FIG. 1 shows a perspective view of a conventional endoscope.

FIG. 1a shows an enlarged view of a circled portion of FIG. 1.

FIG. 1b shows an enlarged view of another circled portion of FIG. 1.

FIG. 2 shows a partial, perspective view of another conventional endoscope.

FIG. 3 shows an exploded, perspective view of an endoscope controlling device according to the present invention.

FIG. 4 shows an exploded, perspective view of a portion of the endoscope controlling device according to the present invention.

FIG. 5 shows a longitudinal cross sectional view of the endoscope controlling device according to the present invention after assembly.

FIG. 6 shows a cross sectional view taken along section line 6-6 of FIG. 5.

FIG. 7 is a view similar to FIG. 6, with a rigid tube of the endoscope controlling device moved along a longitudinal axis.

FIG. 8 is a view similar to FIG. 5, with the rigid tube rotated about the longitudinal axis.

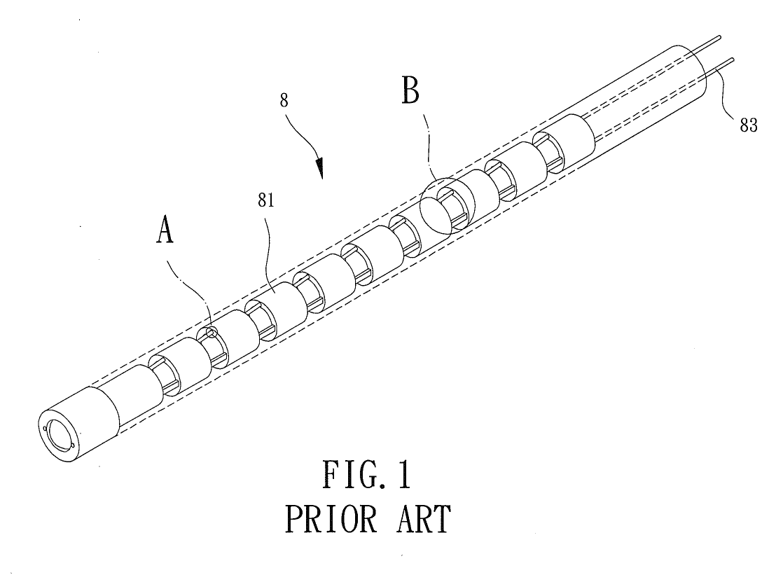

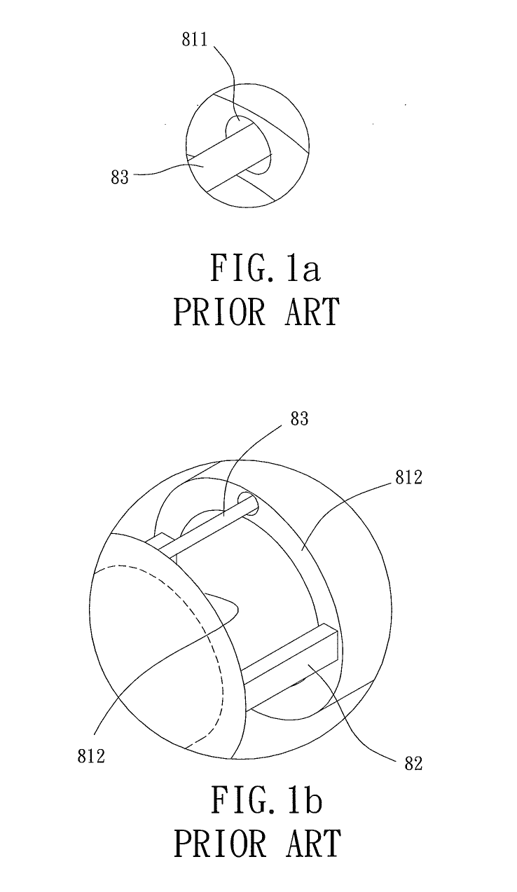

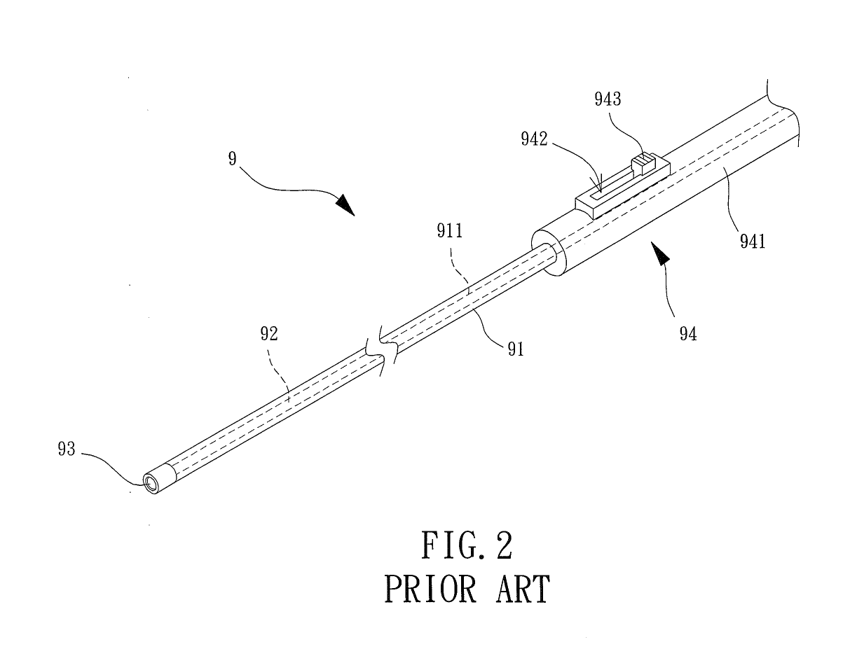

All figures are drawn for ease of explanation of the basic teachings of the present invention only; the extensions of the figures with respect to number, position, relationship, and dimensions of the parts to form the preferred embodiments will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

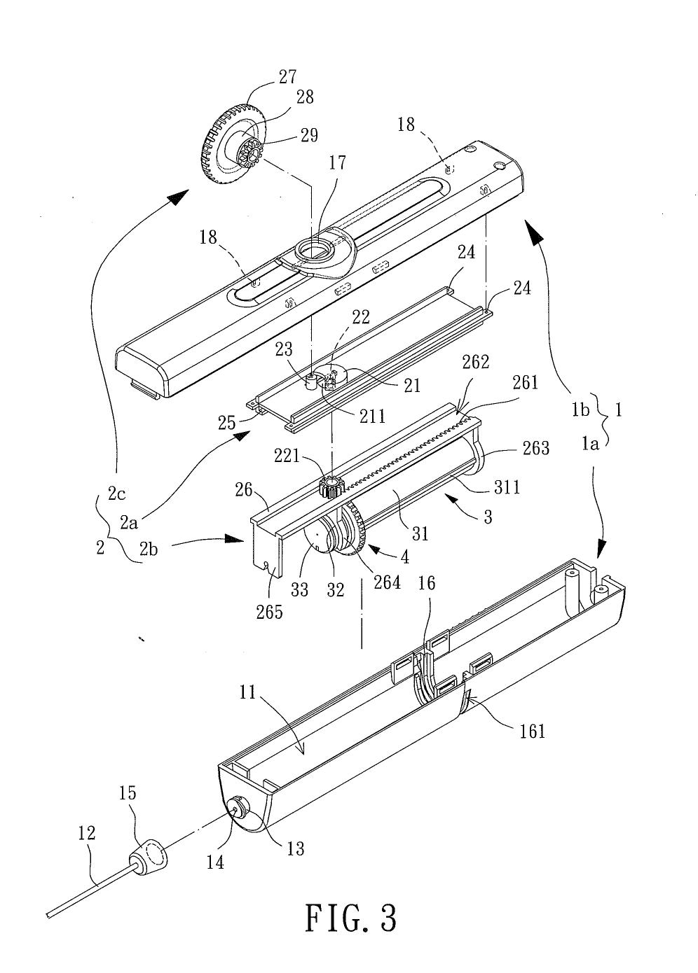

FIGS. 3-5 show an embodiment of an endoscope controlling device according to the present invention. The endoscope controlling device includes a base 1, a linear displacement control module 2, an optical element receiving drum 3, and a rotational movement control module 4. The linear displacement control module 2, the optical element receiving drum 3, and the rotational movement control module 4 are mounted in the base 1.

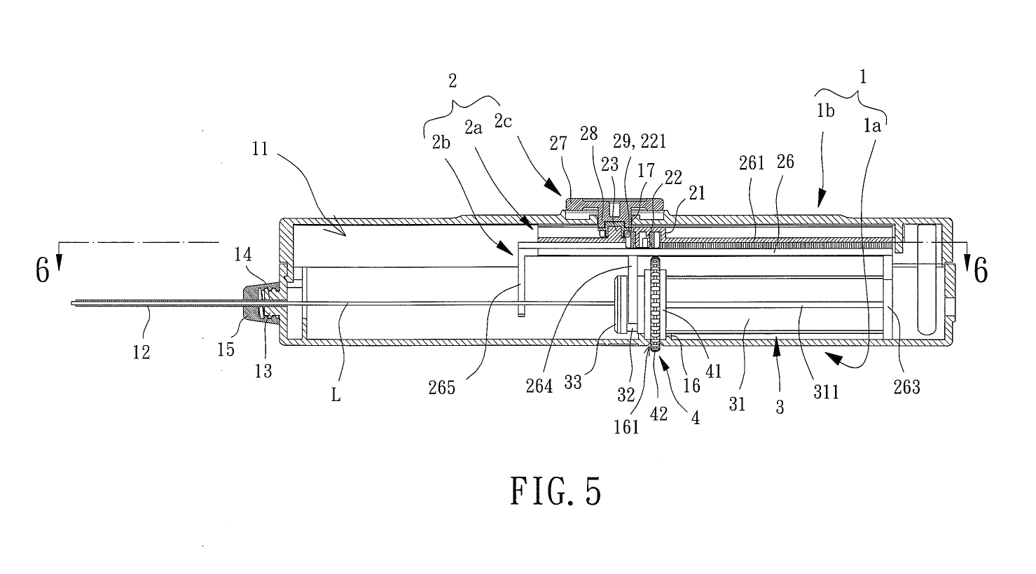

With reference to FIGS. 3 and 5, the base 1 is hollow and includes a compartment 11 for receiving the linear displacement control module 2, the rotational movement control module 4, and the optical element receiving drum 3. In this embodiment, the base 1 includes first and second housings 1a and 1b engaged with each other to form the compartment 11. The first and second housings 1a and 1b can be engaged with each other by any conventional provisions, such as coupling, locking, or gluing.

A rigid tube 12 is provided to an end face of the first housing 1a. Specifically, the rigid tube 12 extends outward from an outer face of the first housing 1a and is in communication with an interior of the first housing 1a. The rigid tube 12 can be integrally formed with the first housing 1a. In the embodiment shown in FIG. 3, a protrusion 13 is formed on the outer face of the first housing 1a and includes a through-hole 14. The rigid tube 12 is coupled to a cover 15. The cover 15 is engaged with the protrusion 13, with the rigid tube 12 in communication with the through-hole 14. The covered area of the rigid tube 12 is increased such that the rigid tube 12 can be more reliably mounted to the end face of the first housing 1a.

A positioning groove 16 is provided in the interior of the first housing 1a for positioning the rotational movement control module 4. In this embodiment, the positioning groove 16 is formed in an inner periphery of the first housing 16 and extends in a radial direction of the base 1. The positioning groove 16 includes a bottom wall having a slot 161 in communication with the outside. A through-hole 17 is formed in a periphery of the second housing 1b. A plurality of fixing portions 18 is provided on an inner periphery of the second housing 1b for coupling with the linear displacement control module 2.

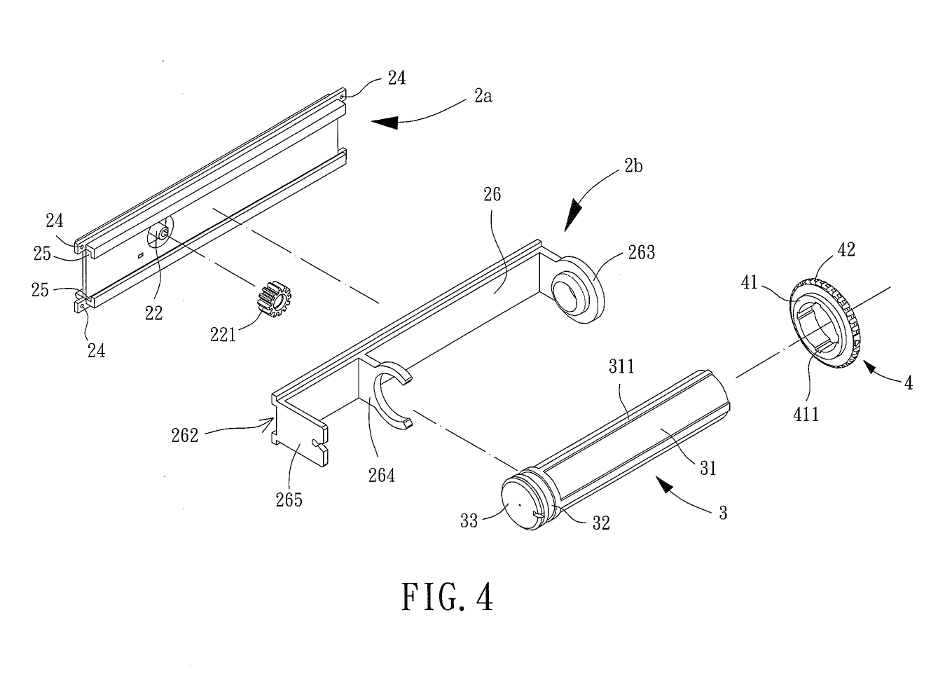

With reference to FIGS. 3 and 4, the linear displacement control module 2 includes a fixed board 2a, a movable board 2b, and a control member 2c. The fixed board 2a is received in the compartment 11 and connected to the base 1. The movable board 2b is slideably mounted on the fixed board 2a. The control member 2c is aligned with the through-hole 17 of the base 1 and controls sliding movement of the movable board 2b relative to the fixed board 2a through manual or electrical control. In this embodiment, the control member 2c controls the sliding movement of the movable board 2b via manual operation. However, other control provisions can be used and appreciated by one skilled in the art.

In the embodiment shown in FIG. 3, the fixed board 2a includes a hollow axle coupling seat 21 on a surface thereof. The axle coupling seat 21 includes a peripheral wall having an opening 211. A first axle coupling portion 22 is received inside the axle coupling seat 21. A driven gear 221 is rotatably mounted to the first axle coupling portion 22. The fixed board 2a further includes a second axle coupling portion 23 located adjacent to the axle coupling seat 21 and aligned with the opening 211. The first axle coupling portion 22 defines a first rotating axis. The second axle coupling portion 23 defines a second rotational axis parallel to the first rotating axis.

The fixed board 2a includes a plurality of positioning portions 24 corresponding to the fixing portions 18 of the second housing 1b. The surface of the fixed board 2a with the second axle coupling portion 23 faces the second housing 1b. The fixed board 2a is fixed to the second hosing 1b by fixing the positioning portions 24 to the fixing portions 18. The second axle coupling portion 23 is aligned with the through-hole 17 of the base 1. The fixed board 2a and the second housings 1b can be engaged with each other by any conventional provisions, such as coupling, locking, or gluing. The fixed board 2a further includes two sliding tracks 25 provided on the other surface of the fixed board 2a. The sliding tracks 25 extend along the longitudinal axis of the base 1. Each sliding track 25 has an opening. The openings of the sliding tracks 25 face each other.

The movable board 2b includes a sliding plate 26 slideably received in the sliding tracks 25 of the fixed board 2a. Thus, the movable board 2b can displace relative to the fixed board 2a. In this embodiment, the sliding plate 26 includes a toothed portion 261 extended along the longitudinal axis of the base 1 and meshed with the driven gear 221. The sliding plate 26 slides in the sliding tracks 25 through the toothed portion 261 when the driven gear 221 rotates, causing linear displacement of the movable board 2b in the compartment 11 along the longitudinal axis of the base 1 relative to the fixed board 2a. Thus, the toothed portion 261 can be a rack engaged on the sliding plate 26. In the embodiment shown, the sliding plate 26 includes a first side having a groove 262 extending along the longitudinal axis of the base 1. The toothed portion 261 is formed on a lateral wall of the groove 262. The driven gear 221 is received in the groove 262 and meshes with the toothed portion 261.

The movable board 2b also positions the optical element receiving drum 3. In this embodiment, the sliding plate 26 further includes a second side opposite to the first side. An abutment portion 263, a retaining portion 264, and a tube holding portion 265 are provided on the second side of the sliding plate 26 along the longitudinal axis of the base 1. The retaining portion 264 is located between the abutment portion 263 and the tube holding portion 265. The optical element receiving drum 3 is retained in place by the retaining portion 264. An end of the optical element receiving drum 3 abuts the abutment portion 263. The other end of the optical element receiving drum 3 extends between the retaining portion 264 and the tube holding portion 265. Thus, the optical element receiving drum 3 and the movable board 2b can move synchronously in the compartment 11 of the base 1.

With reference to FIGS. 3 and 5, the control member 2c is used to control longitudinal movement of the movable board 2b in the compartment 11 along the longitudinal axis of base 1. In this embodiment, the control member 2c includes a disc 27, an axle 28, and a driving gear 29. The disc 27 is mounted to an end of the axle 28. The driving gear 29 is provided on the other end of the axle 28. The disc 27, the axle 28, and the driving gear 29 can be integrally formed. Alternatively, the disc 27, the axle 28, and the driving gear 29 can be manufactured separately and assembled with each other. The axle 28 is extended through the through-hole 17 of the base 1 and mounted around the second axle coupling portion 23 of the fixed board 2a. The disc 27 is exposed outside of the second housing 1b. The driving gear 29 extends through the opening 211 of the axle coupling seat 21 and meshes with the driven gear 221.

With reference to FIGS. 4 and 5, the optical element receiving drum 3 includes a barrel 31, a neck 32, and a head 33. The barrel 31 includes an outer periphery having at least one engagement portion 311 for positioning the rotational movement control module 4. The neck 32 is located between the barrel 31 and the head 33 and has a reduced diameter. The neck 32 is engaged with and positioned by the retaining portion 264 of the sliding plate 26. The barrel 31 receives optical elements, such as a charge-coupled device (CCD) lens assembly, a photosensitive element, and a photosensitive main board. Coupling and operation of the optical elements can be appreciated by persons having ordinary skill in the art.

The rotational movement control module 4 includes a ring 41 and an operative ring 42. The ring 41 includes an inner periphery having at least one engagement section 411. The at least one engagement section 411 is engaged with the at least one engagement portion 311 of the barrel 31, with a number of the at least one engagement section 411 being equal to that of the at least one engagement portion 311. In this embodiment, the at least one engagement section 411 includes a plurality of grooves extending radially in the inner periphery of the ring 41. The at least one engagement portion 311 of the barrel 31 includes a plurality of ribs extending radially outward from the outer periphery of the barrel 31. However, the at least one engagement section 411 can be in the form of ribs, and at least one engagement portion 311 can be in the form of grooves. By engagement between the at least one engagement section 411 and the at least one engagement portion 311, the barrel 3b rotates together with the ring 41 relative to the movable board 2b when the ring 41 rotates. The operative ring 42 is formed on an outer periphery of the ring 41 for manual operation to synchronously rotate the ring 41 and the barrel 31. The ring 41 and the operative ring 42 can be integrally formed. Alternatively, the ring 41 and the operative ring 42 can be manufactured separately, and the operative ring 42 is mounted around the ring 41.

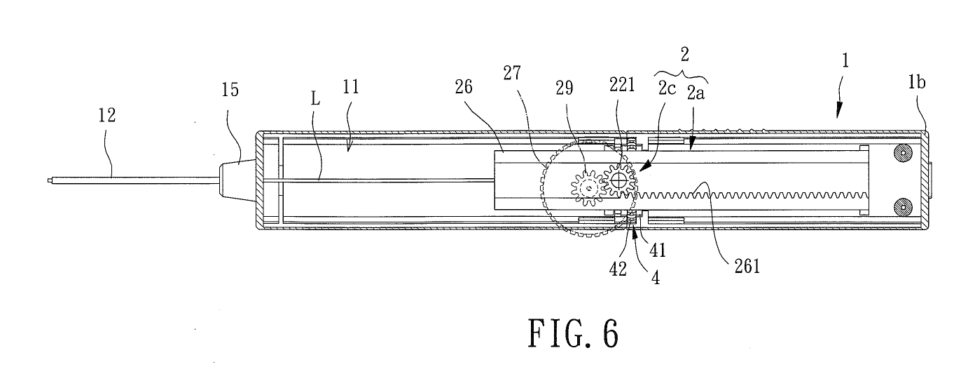

With reference to FIGS. 3 and 5, in assembly, the fixed board 2a is mounted in the interior of the second housing 1b. The sliding plate 26 of the movable board 2b is received in the sliding tracks 25 of the fixed board 2a. The driving gear 29 of the control member 2c extends through the through-hole 17 of the second housing 1b and meshes with the driven gear 221 (FIG. 6). The disc 27 is exposed outside of the second housing 1b. The end of the barrel 31 of the optical element receiving drum 3 abuts the abutment portion 263 of the sliding plate 26. The neck 32 of the optical element receiving drum 3 is engaged with and positioned by the retaining portion 264 of the sliding plate 26. The head 33 of the optical element receiving drum 3 extends between the retaining portion 264 and the tube holding portion 265. The ring 41 of the rotational movement control module 4 is mounted around the barrel 31 and received in the positioning groove 16 of the base 1. The operative ring 42 is extended through the slot 161 and exposed outside of the first housing 1a.

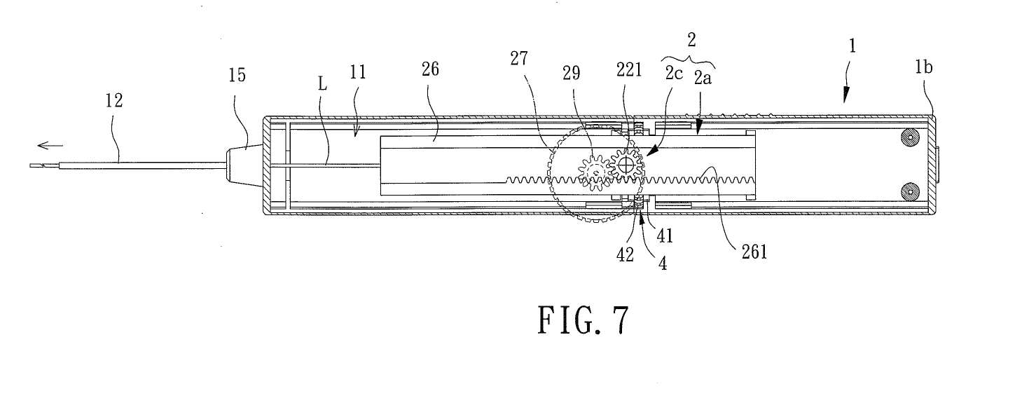

FIGS. 5-8 show use of the endoscope controlling device according to the present invention with an endoscope. In the embodiment shown, the endoscope includes a flexible strip and an image capturing module. The flexible strip is made of a super elastomer that can withstand large deformation and that can restore its original shape before deformation after the load exerting on the flexible strip 2 is released. The flexible strip can be made of a shape memory alloy (such as a nickel-titanium alloy) or a polymer (such as rubber).

The image capturing module includes a photosensitive chip, at least one signal line, and an optical processing unit. The photosensitive chip is mounted in the front end of the flexible strip and electrically connected to the at least one signal line. Two ends of the at least one signal line are respectively connected to the photosensitive chip and the optical processing unit. The optical processing unit is mounted in the barrel 31. The flexible strip and the at least one signal line are covered by the sheath L and extend beyond the head 33 of the optical element receiving drum 3. The sheath L covering the flexible strip and the at least one signal line extends through the tube holding portion 265 and is received in the rigid tube 12. The flexibility of the flexible strip depends on the extended length of the flexible strip outside of the rigid tube 12.

With reference to FIGS. 3, 6, and 7, a user holds the base 1 when in use. If it is desired to adjust the extended length of the sheath L outside of the rigid tube 12, the user operates the control member 2c by his or her fingers to rotate the disc 27 in a direction (such as in a counterclockwise direction). The driving gear 29 synchronously rotates in the counterclockwise direction, causing rotation of the driven gear 221 in a clockwise direction. The sliding plate 26 moves towards a front end of the base 1 via the toothed portion 261. The sliding plate 26 slides in the tracks 25 of the fixed board 2a. Thus, the whole movable board 2b in the compartment 11 and the optical element receiving drum 3 together displace along the longitudinal axis of the base 1. As a result, the sheath L covering the flexible strip and the at least one signal line extends outward of the rigid tube 12. The elastic restoring properties of the flexible strip change the flexibility of the sheath L. The photosensitive chip at the front end of the flexible strip can capture images from different visual angles. The at least one signal line transmits the images captured by the photosensitive chip to the optical processing unit. The optical processing unit proceeds with reception and amplification of the images and converts the images into electronic signals. Then, the electronic signals are transmitted to an external display.

On the other hand, if the disc 27 is rotated in the reverse direction (such as the clockwise direction), the sheath L covering the flexible strip and the at least one signal line retracts into the rigid tube 12. Since the rotational movement control module 4 does not limit the barrel 31 of the optical element receiving drum 3, the optical element receiving drum 3 can displace along the longitudinal axis of the base 1 together with the movable board 2b. At this time, the rotational movement control module 4 remains in the positioning groove 16 of the first housing 1a.

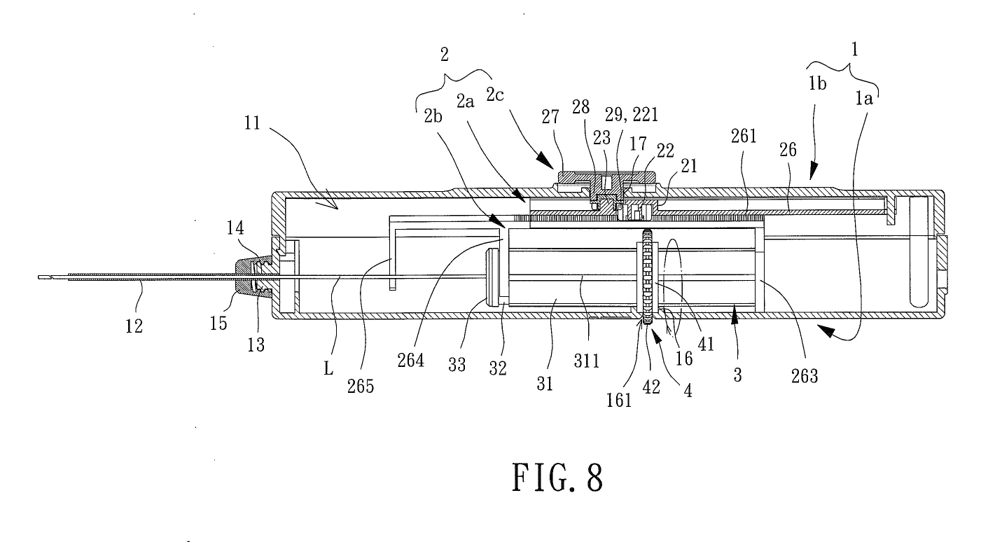

With reference to FIG. 8, if the user intends to adjust the visual angle of the sheath L outside of the rigid tube 12, the user can rotates the operative ring 42 of the rotational movement control module 4, causing synchronous rotation of the optical element receiving drum 3 in the compartment 11 of the base 1 through the ring 41. The sheath L covering the flexible strip and the at least one signal line rotates together with the optical element receiving drum 3 to change the image capturing angle of the outer end of the sheath L outside of the rigid tube 12.

In view of the foregoing, the endoscope controlling device according to the present invention converts the rotational torque into linear displacement, allowing the user to control telescopic movement or rotational movement of the image capturing lens by simply using his or her fingers without changing the position of his or her palm, providing enhanced operational convenience.

The endoscope controlling device according to the present invention allows minor adjustment of the telescopic movement or rotational movement of the image capturing lens through fingers that provide more precision than the wrist, providing enhanced control precision.

The endoscope controlling device according to the present invention can be used in other endoscopes using a flexible strip that can flex automatically, not limited to the endoscope disclosed in Taiwan Patent Application No. 100143086, which can be appreciated by one having ordinary skill in the art.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. An endoscope controlling device comprising:
a base including a compartment, with a rigid tube mounted to an end face of the base and in communication with the compartment, with the base further including an inner periphery having a positioning groove, with the positioning groove including a bottom wall having a slot, with the base further including a periphery having a through-hole;
a linear displacement control module including a control member and a movable board, with the control member aligned with the through-hole of the base, with the control member including a driving gear, with the control member controlling longitudinal movement of the movable board in the compartment along a longitudinal axis of the base via the driving gear;
an optical element receiving drum mounted to the movable board; and
a rotational movement control module mounted around the optical element receiving drum and received in the positioning groove of the base, with a portion of the rotational movement control module extended through the slot and exposed outside of the base.

2. The endoscope controlling device as claimed in claim 1, wherein the linear displacement control module further includes a fixed board received in the compartment and connected to the base, with the fixed board including a surface facing the through-hole of the base, with the movable board slideably mounted to the fixed board.

3. The endoscope controlling device as claimed in claim 2, wherein the surface of the fixed board includes an axle coupling seat having a peripheral wall with an opening, with the axle coupling seat including a first axle coupling portion, with a driven gear rotatably mounted to the first axle coupling portion, with the fixed board further including a second axle coupling portion, with the second axle coupling portion located adjacent to the axle coupling seat and aligned with the opening, with the first axle coupling portion defining a first rotating axis, with the second axle coupling portion defining a second rotational axis parallel to the first rotating axis.

4. The endoscope controlling device as claimed in claim 3, wherein the second axle coupling portion is aligned with the through-hole of the base, with the driving gear extended through the opening of the axle coupling seat and meshed with the driven gear.

5. The endoscope controlling device as claimed in claim 3, wherein the fixed board further includes another surface opposite to the surface, with two sliding tracks provided on the other surface of the fixed board, with each of the two sliding tracks having an opening, with the openings of the two sliding tracks facing each other, with the movable board including a sliding plate, with the sliding plate slideably received in the two sliding tracks of the fixed board.

6. The controlling device endoscope as claimed in claim 5, wherein the sliding plate includes a toothed portion, with the toothed portion meshed with the driven gear.

7. The endoscope controlling device as claimed in claim 6, wherein the sliding plate includes a first side having a groove, with the toothed portion formed on a lateral wall of the groove, with the driven gear received in the groove.

8. The endoscope controlling device as claimed in claim 7, wherein the sliding plate further includes a second side opposite to the first side, with an abutment portion, a retaining portion, and a tube holding portion provided on the second side of the sliding plate along the longitudinal axis of the base, with the retaining portion located between the abutment portion and the tube holding portion, with the optical element receiving drum including a barrel, a neck, and a head, with the abutment portion of the sliding plate abut against an end of the barrel, with the neck engaged with and positioned by the retaining portion of the sliding plate, with the head extending between the retaining portion and the tube holding portion.

9. The endoscope controlling device as claimed in claim 8, wherein the barrel of the optical element receiving drum includes an outer periphery having at least one engagement portion, with the rotational movement control module including a ring and an operative ring, with the ring including an inner periphery having at least one engagement section, with the at least one engagement section engaged with the at least one engagement portion of the barrel, with the operative ring formed on an outer periphery of the ring, with the ring received in the positioning groove of the base, with the operative ring extended through the slot and exposed outside of the base.

10. The endoscope controlling device as claimed in claim 2, wherein the control member further includes a disc and an axle, with the disc mounted to an end of the axle, with the driving gear provided on another end of the axle, with the axle extended through the through-hole of the base and mounted around the second axle coupling portion of the fixed board, with the disc exposed outside of the base.
